(19) United States
(12) Patent Application Publication
(10) Pub. No.: US 2020/0308620 A1
(43) Pub. Date: Oct. 1, 2020

(54) PREDICTING TUMOR SPECIFICITY OF TARGETED THERAPEUTICS USING ATOMIC FORCE MICROSCOPY (AFM)

(71) Applicants: Daxing LIU, Stony Brook, NY (US); Children's Medical Center Corporation, Boston, MA (US)

(72) Inventors: Marsha A. Moses, Brookline, MA (US); Peng Guo, Boston, MA (US); Debra Auguste, Briarcliff Manor, NY (US); Jiang Yang, West Lafayettle, IN (US); Daxing Liu, Stony Brook, NY (US)

(73) Assignee: Children's Medical Center Corporation, Boston, MA (US)

(21) Appl. No.: 16/759,713

(22) PCT Filed: Oct. 26, 2018
(86) PCT No.: PCT/US18/57725

§ 371 Date: Apr. 27, 2020

Related U.S. Application Data
(60) Provisional application No. 62/577,910, filed on Oct. 27, 2017.

(51) Int. Cl. C12Q 1/02; G01Q 60/42

(52) U.S. Cl. CPC C12Q 1/02; C07K 16/30; G01Q 60/42

(57) ABSTRACT

Provided herein are methods of using atomic force microscopy (AFM) to measure the adhesion force between a cell surface target and a ligand (e.g., an antibody) that binds to the cell surface target. Such adhesion force serves as an in vitro metric for predicting the in vivo tumor recognition and/or anti-tumor efficacy of antibody-directed nanomedicine.

### RELATED APPLICATION

This application claims the benefit under 35 U.S.C. § 119(e) to U.S. Provisional Application No. 62/577,910, filed Oct. 27, 2017, and entitled “PREDICTING TUMOR SPECIFICITY OF TARGETED THERAPEUTICS USING ATOMIC FORCE MICROSCOPY (AFM),” the entire contents of which are incorporated herein by reference.

### GOVERNMENT SUPPORT

This invention was made with government support under grant numbers CA185530 and CA174495 awarded by the National Institutes of Health. The Government has certain rights in this invention.

### BACKGROUND

Tumor-targeted therapy is often governed by specific antibody-antigen or ligand-receptor interactions between drug delivery systems and cancer cells. For example, antibody-directed targeting is commonly used to preferentially accumulate nanomedicines in tumor sites. New targets are often identified by measuring statistical increases in mean gene or protein expression in cancer cells relative to normal controls. However, the overexpressed molecules often cannot be used to effectively recognize and target primary tumors and metastatic lesions and, in turn, improve therapeutic efficacy. To date, identifying quantitative metrics for the design of tumor-targeted nanomedicine remains a challenge.

### SUMMARY

The present disclosure is based, at least in part, on the findings that the adhesion force between a cell surface molecule and a ligand (e.g., an antibody) that binds to the cell surface molecule measured by atomic force microscopy (AFM) may be used as an in vitro metric for predicting the in vivo tumor recognition and/or anti-tumor efficacy of antibody-directed nanomedicine.

Accordingly, some aspects of the present disclosure provide methods of identifying a cell surface target, the method comprising: (i) contacting a cell with an atomic force microscopy (AFM) probe functionalized with a ligand that associates with a cell surface molecule of the cell; (ii) dissociating the AFM probe from the cell surface molecule; (iii) measuring an adhesion force between the ligand and the cell surface molecule; and (iv) identifying the cell surface molecule as a cell surface target.

In some embodiments, the cell is a cancer cell. In some embodiments, the cancer cell is a breast cancer cell. In some embodiments, the breast cancer cell is a triple negative breast cancer cell (TNBC).

In some embodiments, the cell surface molecule is a protein, a lipid, or a carbohydrate. In some embodiments, the cell surface molecule is Intercellular Adhesion Molecule 1 (ICAM1). In some embodiments, the ligand is selected from the group consisting of: antibodies, antibody fragments, synthetic peptides, natural ligands, aptamers, small molecules, and live cells.

In some embodiments, the ligand is an ICAM1 antibody. In some embodiments, the ligand is covalently conjugated to the AFM probe.

In some embodiments, the cell is a live cell. In some embodiments, the method is carried out in vitro. In some embodiments, the method is carried out ex vivo.

In some embodiments, the method is carried out repeatedly across the cell surface. In some embodiments, the method further comprises generating a density map of the cell surface molecule on the cell surface.

In some embodiments, the cell surface molecule is identified as a cell surface target if the adhesion force measured in (iii) is above a predetermined value. In some embodiments, the predetermined value is 100 pN.

In some embodiments, the cell surface molecule is identified as a cell surface target if the adhesion force measured in (iii) is 100-500 pN more than a control adhesion force. In some embodiments, the cell surface molecule is identified as a target for in vivo cancer-specific drug delivery if the adhesion force measured in (iii) is at least 400 pN more than a control adhesion force.

In some embodiments, the control adhesion force is the adhesion force measured using an AFM probe functionalized with a non-specific ligand. In some embodiments, the non-specific ligand is a non-specific IgG. In some embodiments, the cell surface molecule is not overexpressed intracellularly or on cell surface.

In some embodiments, the AFM probe is functionalized with a plurality of ligands that each associates with a different cell surface molecule of the cell.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A to 1C. Ranking of 40 cancer-related antigens based on their levels on the TNBC cell surface. (FIG. 1A) Comparative flow cytometric analysis of TNBC target candidate protein levels on the surfaces of MDA-MB-231 (TNBC) and control MCF10A (non-neoplastic) cells. (FIG. 1B) Overexpression of the top ten target candidates from FIG. 1A are quantified for TNBC. The overexpression of each antigen was calculated using the following equation:

Overexpression=Expression_{TNBC}−Expression_{Non-neoplastic}(molecules/cell).

The two-tailed p value was calculated on the basis of surface expression difference between MDA-MB-231 and MCF10 cells. All ten targets were significantly overexpressed in TNBC cells compared to the control. (FIG. 1C) Expression of the top ten target candidates on cell surface of non-neoplastic MCF10A cells.

FIGS. 2A to 2F. AFM measurement of ICAM1 antibody-antigen interaction. (FIG. 2A) Schematic illustration of AFM probing ICAM1 antibody-antigen interaction on live human MDA-MB-231 (TNBC) and MCF10A (non-neoplastic) cells. (FIG. 2B) Adhesion of ICAM1 antibody or non-specific IgG with the MDA-MB-231 cell membrane was detected by AFM using ICAM1 antibody or IgG functionalized AFM tip. (FIG. 2C) Flow cytometric analysis of ICAM1 expression on the MDA-MB-231 cell surface pre- and post-MCD treatment. (FIG. 2D) Adhesion of ICAM1 antibody to MDA-MB-231 and MCF10A cells probed with ICAM1 antibody functionalized AFM cantilevers pre- and post-MCD treatment. (FIGS. 2E and 2F) Adhesion maps of ICAM1 antigen-antibody interaction on MDA-MB-231 (FIG. 2E) and MCF10A (FIG. 2F) cell membrane pre- and post-MCD treatment. (The dashed cycles illustrate the area subjected to high ICAM1 adhesive events). (* p<0.05, ** p<0.01, *** p<0.001).

FIGS. 3A to 3D. In vitro binding and cytotoxicity of ICAM-Dox-LPs. (FIG. 3A) ICAM-RD-LP and IgG-RD-LP binding and uptake in TNBC and normal cell lines were characterized via flow cytometry. Cytotoxicity of ICAM-Dox-LPs in three TNBC cell lines, MDA-MB-231 (FIG. 3B), MDA-MB-436 (FIG. 3C), and MDA-MB-157 (FIG. 3D), was evaluated using a cell viability assay. All cells were treated with ICAM-LP (without Dox), free Dox, and non-specific IgG-Dox-LP as controls. (*p<0.05, *** p<0.001).

FIGS. 4A to 4D. In vivo biodistribution of ICAM1 antibody-directed liposomes. (FIG. 4A) In vivo NIR fluorescent images of mice at different time points after intravenous administration of ICAM-DiR-LPs or IgG-DiR-LPs. (FIG. 4B) Tumor accumulation of ICAM-DiR-LP or IgG-DiR-LP was quantified by fluorescent intensity (n=8 for each group). (FIG. 4C) Ex vivo NIR fluorescent image of tumors and organs (liver, spleen, lung, kidney, heart, and brain) after 48 hours circulation in the body. (FIG. 4D) The biodistribution of ICAM-DiR-LP or IgG-DiR-LP in tumors and different organs was quantified by their fluorescent intensity (n=8 for each group). (NS, non-significant, * p<0.05; *** p<0.001).

FIGS. 5A to 5D. In vivo therapeutic efficacy of ICAM-Dox-LP. (FIG. 5A) Representative images of TNBC tumors treated with PBS (Sham), IgG-Dox-LP, or ICAM-Dox-LP on day 24. (FIG. 5B) Tumor mass in each group (n=7-9 for each group) was quantified (* p<0.05; *** p<0.001). (FIG. 5C) Mouse body weight was monitored during the treatment (n=7-9 for each group). (FIG. 5D) Histology of TNBC tumors. Tumors in different treatment groups were sectioned and stained with H&E and ICAM1 antibody. The scale bar represents 50 μm.

FIG. 6. Flow-chart describing methods and quantification metrics used for predicting the in vivo tumor targeting capacity of ICAM1 antibody-directed liposomes.

FIGS. 7A to 7D. Construction of ICAM-1 antibody-directed, doxorubicin encapsulating liposome (ICAM-Dox-LP) as a TNBC-targeted therapeutic. (FIG. 7A) Schematic illustration of the structure of ICAM-Dox-LP. (FIG. 7B) Hydrodynamic sizes of ICAM-Dox-LP and non-specific IgG-Dox-LP. (FIG. 7C) Stability of ICAM-Dox-LP stored in DMEM with 10% FBS. (FIG. 7D) Release profiles of ICAM-Dox-LP in PBS at pH 7.4 and 5.5.

### DETAILED DESCRIPTION OF CERTAIN EMBODIMENTS

Overexpressed genes or proteins in a diseased cell (e.g., a cancer cell) or on a cell surface as measured by conventional methods (e.g., immunostaining or western blotting) are often used as targets for therapeutics. However, such overexpressed molecules often cannot be used to effectively recognize and target the diseased cell (e.g., a cancer cell) or improve therapeutic efficacy. It is demonstrated herein that the localization, organization, and ligand binding strength of a cell surface molecule (e.g., a protein) play important roles in modulating recognition and targeting of the cell. Atomic force microscopy (AFM) is used herein to measure the adhesion force between a ligand and a cell surface molecule (e.g., a protein). Based on the adhesion force, a cell surface molecule (e.g., a protein) may be identified as a cell surface target. In some embodiments, the cell is a cancer cell and the identified surface target may be used as a therapeutic target for treatment of cancer (e.g., cancer-specific drug delivery system). For example, once a cell surface molecule on a cancer cell is identified as a therapeutic target for the treatment of cancer, ligands for the cell surface molecule may be conjugated to the surface of a delivery vehicle that delivers anti-cancer agents (e.g., a therapeutic nanoparticle such as a liposome). Such delivery vehicle specifically targets the cancer cell and delivers the anti-cancer agents to the cancer cell, thus achieving targeted therapy of the cancer.

Accordingly, some aspects of the present disclosure provide methods of identifying a cell surface target, the method comprising: (i) contacting a cell with an atomic force microscopy (AFM) probe functionalized with a ligand that associates with a cell surface molecule of the cell; (ii) dissociating the AFM probe from the cell surface molecule; (iii) measuring an adhesion force between the ligand and the cell surface molecule; and (iv) identifying the cell surface molecule as a cell surface target.

A “cell surface molecule” is a molecule that is present on the outer surface of a cell. Non-limiting examples of cell surface molecules include proteins (e.g., membrane proteins such as certain cell surface receptors), lipids (e.g., phospholipids or cholesterol), and carbohydrates (e.g., cell surface glycans). “Contacting” means the AFM probe is brought to the cell surface in a distance enough for the ligand on the AFM protein to associate with the cell surface molecule that it targets.

A cell surface molecule may be identified as a cell surface target using the methods described herein. A “cell surface target,” as used herein, refers to a cell surface molecule that may be used to identify the cell. In some embodiments, a cell surface target may be used for targeted delivery of an agent to the cell. For example, the cell may be a cancer cell and a cell surface target on a cancer cell may be used for targeted delivery of anti-cancer agents into the cell (e.g., via a liposome with ligands conjugated on the surface that target the cell surface target, and anti-cancer agents encapsulated in the liposome).

In some embodiment, a cell surface target may be a cell surface molecule (e.g., a protein) that is only present on the surface of one type of cell but not on the surface of other types of cells. In some embodiments, the cell surface target is a cell surface molecule that overexpresses (e.g., the expression level is at least 20% higher) on the surface of one type of cell, compared to other types of cells. In some embodiments, the cell surface target is a cell surface molecule that has an expression level that is at least 20%, at least 30%, at least 40%, at least 50%, at least 60%, at least 70%, at least 80%, at least 90%, at least 100%, 2-fold, 5-fold, 10-fold, 100-fold, 1000-fold, or more on one type of cell or on the surface of one type of cell, compared to other types of cells.

In some embodiments, the cell surface molecule is identified as a cell surface target based on the adhesion force between the cell surface molecule and a ligand. “Adhesion,” as used herein, refers to the tendency of two or more molecules to cling to one another. An “adhesion force,” as used herein, refers to the intermolecular force(s) that cause(s) adhesion of the molecules and can be divided into several types, including, without limitation, chemical adhesion, dispersive adhesion, and electrostatic adhesion. Chemical adhesion occurs when molecules form ionic, covalent, or hydrogen bonds. In dispersive adhesion, molecules are held together by van der Waals forces: the attraction between molecules, each of which has a region of slight positive and negative charge. Electrostatic force occurs when molecules pass electrons to form difference in the electrical charge at the joining. The term “adhesion force” refers to the collective effect of all types of adhesion forces that exist between the molecules.

The adhesion force between molecules (e.g., a cell surface molecule and a ligand) may be measured using known methods in the art. In some embodiments, the adhesion force is measured using atomic force microscopy (AFM). “Atomic force microscopy (AFM)” is a type of scanning probe microscopy (SPM), with demonstrated resolution on the order of fractions of a nanometer, more than 1000 times better than the optical diffraction limit. Information of a surface (e.g., a cell surface) is gathered by “feeling” or “touching” the surface with a mechanical probe. Piezoelectric elements are usually in AFM to facilitate tiny but accurate and precise movements on electronic command, enabling very precise scanning. An important function of AFM is force measuring, e.g., measuring the force between a cell surface molecule and a ligand. One skilled in the art is familiar with AFM and its uses, such as its use in force measuring, e.g., as described in Pierce et al., Langmuir. 1994 September; 10(9): 3217-3221, incorporated herein by reference.

To measure the adhesion force between a cell surface molecule and a ligand, an AFM probe may be functionalized with a ligand that binds to the cell surface molecule. An “AFM probe” is a particular type of scanning probe microscopy (SPM) probe, which is a sharp tip that scans across a surface. Most AFM probes are made from silicon (Si), but borosilicate glass and silicon nitride are also in use. The AFM probes of the present disclosure are functionalized with a ligand that binds to a cell surface molecule (e.g., a cell surface molecule of interest).

“Functionalized,” as used herein, means that the AFM probe surface contains a reactive group (e.g., chemical group) or functional group that may be used to attach (e.g., covalently or non-covalently) a molecule (e.g., a chemical compound or a biological molecular such as a nucleic acid or a polypeptide) to the probe. Methods of functionalizing the AFM probe are known in the art, e.g., as described in Wang et al., Biomaterials, 57 (2015), 161-168, incorporated herein by reference.

A “reactive group” or “functional group” refers to a specific group(s) (moiety(ies)) of atom(s) or bond(s) within a molecule(s) that are responsible for the characteristic chemical reaction(s) of the molecule(s). These terms are used interchangeably herein. One example of such reactive group is a “click chemistry handle.” Click chemistry is a chemical approach introduced in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “Click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgon cycloaddition; thiol-yne addition; imine formation; and Michael additions (e.g., maleimide addition). Non-limiting examples of a click chemistry handle include an azide handle, an alkyne handle, or an aziridine handle. Azide is the anion with the formula N3-. It is the conjugate base of hydrazoic acid (HN3). N3- is a linear anion that is isoelectronic with CO2, NCO—, N2O, NO2+ and NCF. Azide can be described by several resonance structures, an important one being −N═N+=N−. An alkyne is an unsaturated hydrocarbon containing at least one carbon-carbon triple bond. The simplest acyclic alkynes with only one triple bond and no other functional groups form a homologous series with the general chemical formula CnH2n-2. Alkynes are traditionally known as acetylenes, although the name acetylene also refers specifically to C2H2, known formally as ethyne using IUPAC nomenclature. Like other hydrocarbons, alkynes are generally hydrophobic but tend to be more reactive. Aziridines are organic compounds containing the aziridine functional group, a three-membered heterocycle with one amine group (—NH—) and two methylene bridges (—CH2-). The parent compound is aziridine (or ethylene imine), with molecular formula C2H5N.

Other non-limiting, exemplary reactive groups include: acetals, ketals, hemiacetals, and hemiketals, carboxylic acids, strong non-oxidizing acids, strong oxidizing acids, weak acids, acrylates and acrylic acids, acyl halides, sulfonyl halides, chloroformates, alcohols and polyols, aldehydes, alkynes with or without acetylenic hydrogen amides and imides, amines, aromatic, amines, phosphines, pyridines, anhydrides, aryl halides, azo, diazo, azido, hydrazine, and azide compounds, strong bases, weak bases, carbamates, carbonate salts, chlorosilanes, conjugated dienes, cyanides, inorganic, diazonium salts, epoxides, esters, sulfate esters, phosphate esters, thiophosphate esters borate esters, ethers, soluble fluoride salts, fluorinated organic compounds, halogenated organic compounds, halogenating agents, aliphatic saturated hydrocarbons, aliphatic unsaturated hydrocarbons, hydrocarbons, aromatic, insufficient information for classification, isocyanates and isothiocyanates, ketones, metal hydrides, metal alkyls, metal aryls, and silanes, alkali metals, nitrate and nitrite compounds, inorganic, nitrides, phosphides, carbides, and silicides, nitriles, nitro, nitroso, nitrate, nitrite compounds, organic, non-redox-active inorganic compounds, organometallics, oximes, peroxides, organic, phenolic salts, phenols and cresols, polymerizable compounds, quaternary ammonium and phosphonium salts, strong reducing agents, weak reducing agents, acidic salts, basic salts, siloxanes, inorganic sulfides, organic sulfides, sulfite and thiosulfate salts, sulfonates, phosphonates, organic thiophosphonates, thiocarbamate esters and salts, and dithiocarbamate esters and salts. In some embodiments, the reactive group is a carboxylic acid group. In some embodiments, the reactive group is an amine group. One skilled in the art is familiar with methods of attaching functional groups on AFM probes. Functionalized AFM probes are also commercially available, e.g., from NanoAndMore USA (Watsonville, Calif.).

In some embodiments, a crosslinker is tethered to the reactive group on the functionalized AFM probe. For example, in some embodiments, the reactive group on the functionalized AFM tip is an amine to which a crosslinker containing a —NHS group is tethered. In some embodiments, the crosslinker is an acetal-polyethylene glycol-NHS (acetal-PEG-NHS) and the acetal group on the crosslinker may be used to further attach a ligand (e.g., a protein ligand such as an antibody). One skilled in the art is familiar with crosslinkers that may be used.

Any ligands (e.g., a protein ligand) may be attached to the AFM probe. The attachment can be, for example, via a direct or indirect (e.g., via a linker) covalent linkage or via non-covalent interactions. A “ligand,” as used herein, refers to a molecule that specifically associates with and forms a complex with another molecule. In some embodiments, the ligand binds a cell surface molecule (e.g., a cell surface protein such as a cell surface receptor). The binding of a ligand to the cell surface molecule may be via intermolecular forces, such as ionic bonds, hydrogen bonds and Van der Waals forces. Ligands include, without limitation substrates, inhibitors, activators, antibodies, and neurotransmitters. The rate of binding is called affinity (KD) and reflects the tendency or strength of the effect of binding. Binding affinity is actualized not only by target-ligand interactions, but also by solvent effects that can play a dominant, steric role which drives non-covalent binding in solution. The solvent provides a chemical environment for the ligand and receptor to adapt, and thus accept or reject each other as partners.

Suitable ligands that may be attached to the AFM tip include, without limitation: antibodies or antibody fragments, inhibitory peptides including peptides derived from natural proteins and synthetic peptides, natural inhibitory ligands, small molecules (e.g., small molecule inhibitors), aptamers, and live cells.

“Antibodies” and “antibody fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

An “antibody fragment” for use in accordance with the present disclosure contains the antigen-binding portion of an antibody. The antigen-binding portion of an antibody refers to one or more fragments of an antibody that retain the ability to specifically bind to an antigen (e.g., a cell surface molecule). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. In some embodiments, the cell surface molecule of the present disclosure, in some embodiments, is Intercellular adhesion molecule 1 (ICAM1). ICAM1 antibodies are known to those skilled in the art and are commercially available (e.g., from Santa Cruz or Abcam).

“Inhibitory peptides” refers to peptides that specifically binds to a cell surface molecule and inhibits the cell surface molecule (e.g., inhibits signaling by the cell surface molecule). For example, the cell surface molecule of the present disclosure, in some embodiments, is ICAM1. Peptides that are derived from the ICAM1 binding portion of proteins that binds to ICAM1 (e.g., integrin) may be used as an inhibitory peptide in accordance with the present disclosure. Synthetic peptides may be obtained using methods that are known to those skilled in the art. Synthetic peptides that inhibit ICAM1 function are known in the art, e.g., as described in Zimmerman et al., Chem Biol Drug Des. 2007 October; 70(4):347-53. Epub 2007, incorporated herein by reference.

A “natural ligand” is a ligand that exists in nature. The present disclosure encompass natural ligands for proteins that specifically express or overexpress on the surface of a cell targeted by the nanoparticles described herein (e.g., a cancer cell). In some embodiments, the natural ligands of the present disclosure inhibit the signaling of the cell surface molecule (e.g., ICAM1).

An “aptamer” refers to an oligonucleotide or a peptide molecule that binds to a specific target molecule. Aptamers are usually created by selecting them from a large random sequence pool. One skilled in the art is familiar with methods of designing and generating aptamers.

A “small molecule,” as used herein, refers to a molecule of low molecular weight (e.g., <900 daltons) organic or inorganic compound that may function in regulating a biological process. Non-limiting examples of a small molecule include lipids, monosaccharides, second messengers, other natural products and metabolites, as well as drugs and other xenobiotics.

A “lipid” refers to a group of naturally occurring molecules that include fats, waxes, sterols, fat-soluble vitamins (such as vitamins A, D, E, and K), monoglycerides, diglycerides, triglycerides, phospholipids, and others. A “monosaccharide” refers to a class of sugars (e.g., glucose) that cannot be hydrolyzed to give a simpler sugar. Non-limiting examples of monosaccharides include glucose (dextrose), fructose (levulose) and galactose. A “second messenger” is a molecule that relays signals received at receptors on the cell surface (e.g., from protein hormones, growth factors, etc.) to target molecules in the cytosol and/or nucleus. Nonlimiting examples of second messenger molecules include cyclic AMP, cyclic GMP, inositol trisphosphate, diacylglycerol, and calcium. A “metabolite” is a molecule that forms as an intermediate product of metabolism. Non-limiting examples of a metabolite include ethanol, glutamic acid, aspartic acid, 5′ guanylic acid, Isoascorbic acid, acetic acid, lactic acid, glycerol, and vitamin B2. A “xenobiotic” is a foreign chemical substance found within an organism that is not normally naturally produced by or expected to be present within. Non-limiting examples of xenobiotics include drugs and antibiotics.

In some embodiments, the cell surface molecule is ICAM1. Small molecule ligands of ICAM1 are known to those skilled in the art. Non-limiting, exemplary small molecule ligands for ICAM1 include metadichol, methimazole, and silibinin.

In some embodiments, a plurality of ligands (e.g., ligands that bind to different cell surface molecules) may be conjugated to the AFM probe, each ligand targeting a different cell surface protein. In some embodiments, 2-10 cell surface proteins are targeted by the ligands conjugated to the AFM probe. For example, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 cell surface proteins may be targeted. In some embodiments, 1, 2, 3, 4, 5, 6, 7, 8, 9, 10, or more cell surface proteins are targeted.

In some embodiments, the ligand conjugated to the AFM tip is a live cell. Cells that may be conjugated to the AFM tip include, without limitation, to human or mouse cancer cells, stem cells, endothelial cells, white blood cells, red blood cells, and platelets. Methods of conjugating a live cell to the AFM tip are known in the art, e.g., as described in an instruction manual published by JPK Instructions AG (California, USA), titled “Attaching microspheres to cantilevers using the NanoWizard Life Science State and AFM head;” or as described herein Hsiao et al., Angew Chem Int Ed Engl. 2008; 47(44): 8473-8477, incorporated herein by reference.

The methods of identifying a cell surface target described herein comprises contacting a cell with the AFM probe functionalized with a ligand that associates with a cell surface molecule, such that the ligand associates with the cell surface molecule, and dissociating the AFM probe from the cell surface molecule. The term “associate” refers to the binding of two entities (e.g., the ligand and the cell surface molecule). Two entities (e.g., two proteins) are considered to associate with each other when the affinity (KD) between them is <10⁻³M, <10⁻⁴ M, <10⁻⁵ M, <10⁻⁶ M, <10⁻⁷ M, <10⁻⁸ M, <10⁻⁹ M, <10⁻¹⁰ M, <10⁻¹¹ M, or <10⁻¹² M. One skilled in the art is familiar with how to assess the affinity between two entities (e.g., two proteins). In some embodiments, the association between two molecules (e.g., the cell surface molecule and the ligand) may be caused by ionic interactions, van der Waals forces, or hydrogen bonds. The term “dissociate” means to separate two molecules (e.g., the ligand and the cell surface molecule) that are associated such that they are no longer associated (e.g., such that the distance between the two molecules is far enough to eliminate the molecular interaction(s) between them). Two molecules with stronger adhesion force to each other are more difficult to dissociate, while two molecules with weaker adhesion force to each other are easier to dissociate. The functionalized AFM probe can measure and quantify measure (using piconewton (pN) as units) the adhesion force between the cell surface molecule and the ligand.

In some embodiments, the methods of measuring the adhesion force is carried out multiple times across the cell surface. For example, the functionalized AFM probe can repeat the associate-dissociate steps at different locations of the cell surface, each repeat giving rise to an adhesion force and a measurement. The steps may be repeated as many times as needed, e.g., 1-10⁵ times, or more. In some embodiments, a density map of the cell surface molecule on the cell surface is generated. As demonstrated herein, the distribution of a cell surface molecule on a cell surface is not uniform, but is rather heterogeneously organized on the cell surface. As such, on some spots of the cell surface where the molecule is concentrated, adhesion force “hot spots” can form and a density map depicting such hot spots may be generated accordingly.

In some embodiments, the adhesion force between the cell surface molecule and the ligand measured by AFM is 10-1000 pN. For example, the adhesion force between the cell surface molecule and the ligand measured by AFM may be 10-1000, 10-900, 10-800, 10-700, 10-600, 10-500, 10-400, 10-300, 10-200, 10-100, 10-90, 10-80, 10-70, 10-60, 10-50, 10-40, 10-30, 10-20, 20-1000, 20-900, 20-800, 20-700, 20-600, 20-500, 20-400, 20-300, 20-200, 20-100, 20-90, 20-80, 20-70, 20-60, 20-50, 20-40, 20-30, 30-1000, 30-900, 30-800, 30-700, 30-600, 30-500, 30-400, 30-300, 30-200, 30-100, 30-90, 30-80, 30-70, 30-60, 30-50, 30-40, 40-1000, 40-900, 40-800, 40-700, 40-600, 40-500, 40-400, 40-300, 40-200, 40-100, 40-90, 40-80, 40-70, 40-60, 40-50, 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 pN. In some embodiments, the adhesion force between the cell surface molecule and the ligand measured by AFM is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 pN.

The methods of identifying a cell surface target described herein comprises identifying the cell surface molecule as a cell surface target. In some embodiments, a cell surface molecule is identified as a cell surface target when the adhesion force between the cell surface molecule and a ligand measured by AFM is above a predetermined value of adhesion force. In some embodiments, the predetermined value of adhesion force is 70-120 pN. For example, the predetermined value of adhesion force may be 70-120, 70-110, 70-100, 70-90, 70-80, 80-120, 80-110, 80-100, 80-90. 90-120, 90-110, 90-100, 100-120, 100-110, or 110-120 pN. In some embodiments, the predetermined value of adhesion force is 85-110 pN. For example, the predetermined value of adhesion force may be 85-110, 85-105, 85-100, 85-95, 85-90, 90-110, 90-105, 90-100, 90-95, 95-110, 95-105, 95-100, 100-110, 100-105, or 105-110 pN. In some embodiments, the predetermined value is 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pN.

In some embodiments, a cell surface molecule is identified as a cell surface target when the adhesion force between the cell surface molecule and a ligand measured by AFM is at least 100 pN more than a control adhesion force. In some embodiments, a cell surface molecule is identified as a cell surface target when the adhesion force between the cell surface molecule and a ligand measured by AFM is at least 100 pN, at least 150 pN, at least 200 pN, at least 250 pN, at least 300 pN, at least 350 pN, at least 400 pN, at least 450 pN, at least 500 pN, or more than a control adhesion force. In some embodiments, a cell surface molecule is identified as a cell surface target when the adhesion force between the cell surface molecule and a ligand measured by AFM is 100-500 pN more than a control adhesion force. For example, a cell surface molecule is identified as a cell surface target when the adhesion force between the cell surface molecule and a ligand measured by AFM may be 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500 pN more than a control adhesion force. In some embodiments, a cell surface molecule is identified as a cell surface target when the adhesion force between the cell surface molecule and a ligand measured by AFM is 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 pN or more than a control adhesion force. In some embodiments, a cell surface molecule is identified as a cell surface target when the adhesion force between the cell surface molecule and a ligand measured by AFM is 427 pN more than a control adhesion force.

In some embodiments, the methods described herein are used on a cancer cell, e.g., to identify a cell surface target on the cancer cell. In some embodiments, the cell surface target on the cancer cell, identified using the methods described herein, is used as a target for in vivo cancer-specific drug delivery. “In vivo cancer-specific drug delivery” refers to therapeutic methods for treating cancer, where the anti-cancer drugs are specifically delivered to the cancer cells but not to normal cells. In some instances, such cancer specific drug delivery systems specifically recognize and/or bind to cell surface molecules that are specific to cancer cells. In some embodiments, the recognition and/or binding of the cell surface molecules that are specific to tumor cells is via ligands (e.g., antibodies). For example, ligands that specifically recognize and bind to a cell surface molecule may be conjugated to a nanoparticle (e.g., liposome) encapsulating anti-cancer agents. The cancer-specific drug delivery systems can be administered to a subject having cancer and can target cancer cells in vivo. As such, the identification of new cell surface target(s) for in vivo cancer-specific drug delivery system is also within the scope of the present disclosure. It was demonstrated herein that the adhesion force between a ligand and a cell surface molecule measured using the methods described herein correlates with the in vivo cancer recognition and drug delivery efficiency of the cancer-specific drug delivery systems. Adhesion force between a cell surface molecule and a ligand measured by AFM may be used to predict the specificity and drug-delivery efficiency of a cancer-specific drug delivery system (e.g., a liposome) that targets the cell surface molecule.

In some embodiments, a cell surface molecule is identified as a target for in vivo cancer-specific drug delivery if the adhesion force measured using the methods described herein is at least 400 pN more than a control adhesion force. For example, a cell surface molecule is identified as a target for in vivo cancer-specific drug delivery if the adhesion force measured using the methods described herein is at least 400 pN, at least 500 pN, at least 600 pN, at least 700 pN, at least 800 pN, at least 900 pN, at least 1000 pN or more than a control adhesion force. In some embodiments, a cell surface molecule is identified as a target for in vivo cancer-specific drug delivery if the adhesion force measured using the methods described herein is 400 pN, 500 pN, 600 pN, 700 pN, 800 pN, 900 pN, 1000 pN, or more than a control adhesion force.

A “control adhesion force” refers to the adhesion force measured between a cell surface molecule and a non-specific ligand. A “non-specific ligand” is a ligand that does not bind to the cell surface molecule, e.g., the affinity between the non-specific ligand and the cell surface molecule is more than 10⁻³M. In some embodiments, the non-specific ligand is a non-specific immunoglobulin G (IgG). A “non-specific IgG” may be an IgG that does not specifically associate with a particular cell surface molecule, or an IgG that does not have any binding specificity.

In some embodiments, a cell surface molecule identified as a cell surface target based on the adhesion force between the cell surface molecule and a ligand using is not overexpressed in the cell. “Not overexpressed” means that the expression level of the cell surface molecule in the cell is less than 20% more than its expression level in a different cell type. For example, the expression level of the cell surface molecule identified as a cell surface target may be less than 20% more, less than 15% more, less than 10%, less than 5% more, less than 1% more than its expression level in a different cell type. In some embodiments, the expression level of the cell surface molecule identified as a cell surface target is equal or less than its expression level in a different cell type.

The drug delivery efficiency of a cancer-specific drug delivery system may be enhanced if the system targets a cell surface molecule identified as a target for in vivo cancer-specific drug delivery using the methods described herein. In some embodiments, the drug delivery efficiency is enhanced by at least 20%, at least 30%, at least 40%, at least 50%, at least 60%, at least 70%, at least 80%, at least 90%, at least 100%, at least 2-fold, at least 3-fold, at least 4-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 1000-fold, or more, compared to that of a drug delivery system targeting a different cell surface molecule. In some embodiments, the drug delivery efficiency is enhanced by 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 60-fold, 70-fold, 80-fold, 90-fold, 100-fold, 1000-fold, or more, compared to that of a drug delivery system targeting a different cell surface molecule. In some embodiments, the efficiency of the cancer-specific drug delivery system is indicated by the accumulation of the anti-cancer agents in cancer cells.

In some embodiments, the methods of the present disclosure may be carried out in vitro (e.g., on the surface of a cultured cell) or ex vivo (e.g., on the surface of a cell isolated from a subject). A subject shall mean a human or vertebrate animal or mammal including but not limited to a rodent, e.g., a rat or a mouse, dog, cat, horse, cow, pig, sheep, goat, turkey, chicken, and primate, e.g., monkey.

In some embodiments, the cell is a live cell. In some embodiments, the cell is a cancer cell. For the purpose of the present disclosure, cancer encompasses benign tumor and malignant cancer. The phrases “tumor” and “cancer” are used interchangeably herein. The cancer cell may be a primary or metastatic cancer cell. Cancers include, but are not limited to, adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, biliary tract cancer, osteosarcoma, fibrous histiocytoma, brain cancer, brain stem glioma, cerebellar astrocytoma, malignant glioma, glioblastoma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, acute lymphocytic and myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, choriocarcinoma, hematological neoplasm, adult T-cell leukemia, lymphoma, lymphocytic lymphoma, stromal tumors and germ cell tumors, or Wilms tumor. In some embodiments, the cancer is lung cancer, breast cancer, prostate cancer, colorectal cancer, gastric cancer, liver cancer, pancreatic cancer, brain and central nervous system cancer, skin cancer, ovarian cancer, leukemia, endometrial cancer, bone, cartilage and soft tissue sarcoma, lymphoma, neuroblastoma, nephroblastoma, retinoblastoma, or gonadal germ cell tumor.

In some embodiments, the cancer is breast cancer. In some embodiments, the cancer is triple-negative breast cancer (TNBC). In some embodiments, the present disclosure provide measuring the adhesion force between a cell surface molecule on TNBC, the Intercellular Adhesion Molecule 1 (ICAM1). ICAM1 is a member of the super-immunoglobulin family of molecules. Members of this superfamily are characterized by the presence of one or more Ig homology regions, each consisting of a disulfide-bridged loop that has a number of anti-parallel β-pleated strands arranged in two sheets. Three types of homology regions have been defined, each with a typical length and having a consensus sequence of amino acid residues located between the cysteines of the disulfide bond. (Williams, A. F. et al., Ann. Rev. Immunol. 6:381-405 (1988); Hunkapillar, T. et al., Adv. Immunol. 44:1-63 (1989)). ICAM1 is a cell surface glycoprotein of 97-114 kd. ICAM1 has 5 Ig-like domains. Its structure is closely related to those of the neural cell adhesion molecule (NCAM) and the myelin-associated glycoprotein (MAG) (e.g., as described Simmons, D. et al., Nature 331:624-627 (1988); Staunton, D. E. et al., Cell 52:925-933 (1988); Staunton, D. E. et al., Cell 61243-254 (1990), herein incorporated by reference). ICAM has previously been shown to overexpression on TNBC cells and has been characterized as a molecular target for TNBC (e.g., as described in Guo et al., PNAS, vol. 111, no. 41, pages 14710-14715, 2014; and Guo et al., Theranostics, Vol. 6, Issue 1, 2016, incorporated herein by reference). As such, in some embodiments, the AFM probe is functionalized with an ICAM1 ligand, e.g., an ICAM1 antibody. ICAM1 antibodies are known to those skilled in the art and are commercially available (e.g., from Santa Cruz or Abcam).

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: Antibody-Antigen Interaction on Live Cancer Cells Predicts Tumor Recognition for Nanomedicine

Tumor-targeted therapy is often governed by specific antibody-antigen or ligand-receptor interactions between drug delivery systems and cancer cells. For example, antibody-directed targeting is commonly used to preferentially accumulate nanomedicines in tumor sites¹⁻³. It requires a tumor-specific antibody or ligand to be conjugated to a drug delivery system to recognize and bind antigen on receptor-overexpressing tumors. To date, MM-302 (human epidermal growth factor receptor 2 (HER2) antibody-conjugated liposomal doxorubicin), an anti-cancer liposome, has demonstrated promising clinical benefits for HER2-positive metastatic breast cancer patients by significantly improving median progression free survival by 7.6 months with an overall response rate of 11%⁴. However, unlike HER2-positive breast cancer, no clinically effective therapeutic target has been identified for TNBC, a highly malignant form of breast cancer defined by the absence of HER2, estrogen receptor (ER), and progesterone receptor (PR). Identification of a TNBC target therefore is pivotal for the development of tumor-targeted therapeutics and subsequent positive patient prognosis⁵⁻⁹.

Most targeted therapeutic studies focus on the identification of overexpressed genes or proteins in cancer cells. A central question is whether these overexpressed molecules can be used to effectively recognize and target primary tumors and metastatic lesions and, in turn, improve therapeutic efficacy. However, quantifying the overexpression of a molecular target in cancer cells relative to normal cells alone may not be sufficient to answer this question, given that overexpression does not always translate into specific targeting in vivo. The localization, organization, and ligand binding strength of a molecular target also play critical roles in modulating tumor recognition and targeting. Acquisition of this information is often limited by conventional assays that evaluate the average levels of a target (e.g., PCR, western blot) or that probe the ligand-target interaction in the absence of living cells (e.g. immunoprecipitation).

It has been previously demonstrated that AFM is a powerful tool to directly detect antibody-antigen interactions on live cell surfaces^{10,11}. Given the importance of antibody-antigen interactions for tumor recognition, it is hypothesized that the in vitro antibody-antigen binding force quantified using AFM could be used as a quantitative metric to predict the in vivo tumor recognition of its antibody-directed nanomedicine. To test this idea, AFM was used to quantitatively map antibody binding events of ICAM1, a recently discovered TNBC target^{12,13}, on live TNBC and non-neoplastic cell surfaces, and subsequently calculated the tumor-recognition affinity. In this study, proof-of-principle evidence that correlates the in vitro ICAM1 antibody-antigen binding force with the in vivo tumor recognition and therapeutic efficacy of ICAM1 antibody-directed liposomes is presented.

Identifying quantitative metrics for the design of tumor-targeted nanomedicine remains a challenge. New targets are often identified by measuring statistical increases in mean gene or protein expression in cancer cells relative to normal controls. Herein, atomic force microscopy (AFM) is utilized to directly measure the antibody-antigen binding force of a cancer target on live breast cancer cell surfaces and, for the first time, used it as a novel in vitro metric for predicting the in vivo tumor recognition of antibody-directed nanomedicine. The AFM results outlined herein reveal that the antibody against ICAM1, a recently identified triple negative breast cancer (TNBC) target, exhibited a statistically stronger antibody-antigen binding force on live TNBC cells than on non-neoplastic mammary epithelial cells. Moreover, using an in vivo orthotopic model, the first proof-of-principle evidence that the in vitro ICAM1 antibody-antigen binding force more precisely correlates with the in vivo tumor accumulation and therapeutic efficacy of ICAM1 antibody-directed liposomes than the ICAM1 gene and surface protein overexpression levels, two established quantitative metrics for cancer targets, is provided. Taken together, this study demonstrates that AFM may be a useful tool for predicting in vivo tumor specificity of antibody-directed nanomedicines.

The application of this AFM-based biomechanical measurement is not limited to the study of antibody-antigen interactions but can be applied to a variety of biological molecular interactions, such as small molecule-protein interactions and live cell-cell interactions. In the case of small molecules, the small molecules can be attached to the surface of the AFM tip via either covalent or non-covalent conjugation and the obtained small molecule attached AFM tip can be used to quantitatively map and measure the small molecule-protein interaction on live cells, as it did for antibody-antigen interactions. This application has the important potential to be used in this way for small molecule drug discovery.

In the case of live cell-cell interactions, one live cell can be attached to the surface of AFM tip and this single, attached live cell can be used to quantitatively map and measure the live cell-cell interaction on other live cells. This approach has significant potential in the investigation of live immune cell-tumor cell interactions as needed in cancer immunotherapy. The quantified binding force of the live immune cell-tumor cell interaction has the potential to predict the in vivo efficacy of cancer immunotherapy.

### Assessment of Cell Surface Antigens Overexpressed in TNBC

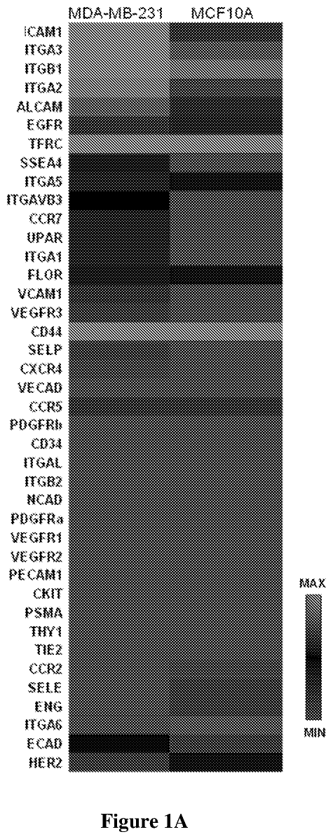

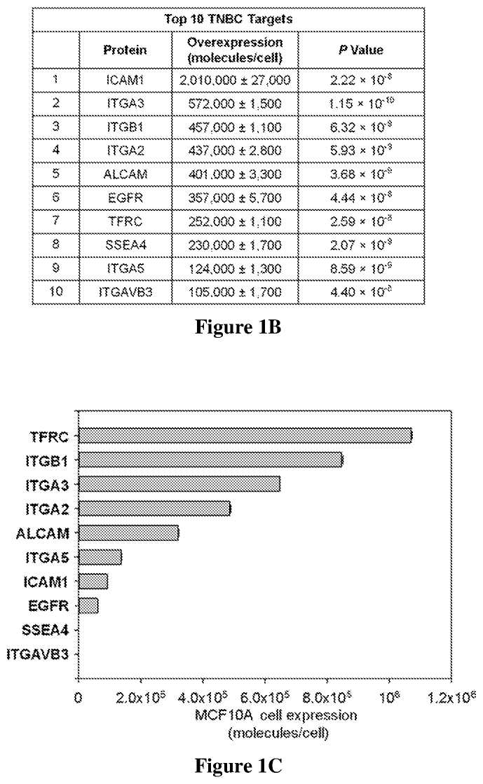

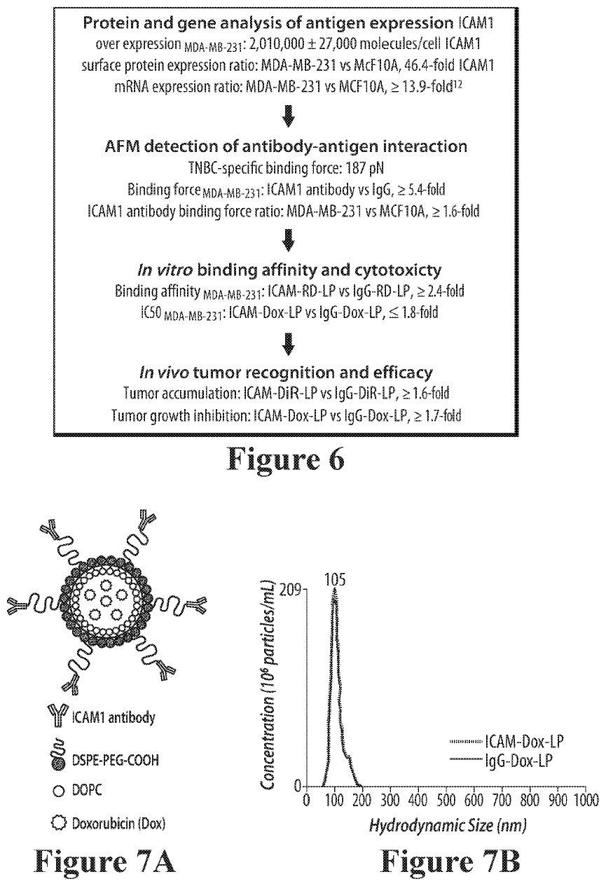

It has been previously demonstrated that ICAM1 levels are significantly elevated in human TNBC tissues and cell lines, suggesting it as a novel TNBC target^{12,13}. However, because no quantitative comparison between ICAM1 and other reported TNBC targets including epidermal growth factor receptor (EGFR)¹⁴, plasminogen activator urokinase receptor (PLAUR)^{15,16}, CD44¹⁷ and transferrin receptor (TFRC)¹⁸ has been conducted, it remains unknown which cancer target is optimal for TNBC-targeting nanomedicine. In this study, an unbiased and quantitative assessment of a panel of 40 cancer-related cell surface antigens on TNBC cells (Table 1) was performed. Protein levels on the surface of human TNBC MDA-MB-231 cells and non-neoplastic MCF10A cells were quantified by flow cytometric analysis (FIG. 1A). TNBC target candidates were ranked according to their overexpression levels on MDA-MB-231 cells relative to MCF10A cells. Twenty-two of 40 examined antigens were upregulated on MDA-MB-231 cells; the top ten TNBC-overexpressed antigens are listed in FIG. 1B. ICAM1 emerged as the most significantly overexpressed molecule, with respect to the control, among the 40 tested candidates. ICAM1 protein was expressed at a level that is 46.4-fold higher on MDA-MB-231 cells than MCF10A cells. The cell surface densities of the top ten TNBC-overexpressed antigens on non-neoplastic MCF10A cells were further compared (FIG. 1C). ICAM1 was expressed at a significantly lower level on MCF10A cells relative to other highly overexpressed TNBC targets such as integrin alpha 3 (ITGA3) and integrin beta 1 (ITGB1). TFRC and CD44, two broadly-used cancer targets in nanomedicine, were identified as being unsuitable for TNBC-targeting due to their high expression on non-neoplastic MCF10A cells (FIG. 1A). Given its tumor specificity and overexpression levels, it is postulated that ICAM1 is a key target for TNBC-targeted nanomedicine, and ICAM1 was focused on to investigate its antibody-antigen interactions on live human TNBC cells and the implications for in vivo TNBC-targeted drug delivery. It is worth noting that the gene and surface protein overexpression levels of ICAM1 on MDA-MB-231 cells, two established quantitative metrics for defining cancer target, are 13.9¹² and 46.4-folds over non-neoplastic controls (MCF10A cells), respectively (FIG. 6).

TABLE S1
List of cancer-related epitope symbols

| Symbol | Description |
|---|---|
| ICAM1 | Intercellular adhesion molecule 1 |
| ITGA3 | Integrin, alpha 3 |
| ITGB1 | Integrin, beta 1 |
| ITGA2 | Integrin, alpha 2 |
| ALCAM | Activated leukocyte cell adhesion molecule |
| EGFR | Epidermal growth factor receptor |
| TFRC | Transferrin receptor |
| SSEA4 | Stage specific embryonic antigen 4 |
| ITGA5 | Integrin, alpha 5 |
| ITGAVB3 | Integrin, alpha V beta 3 |
| CCR7 | Chemokine (C-C motif) receptor 7 |
| PLAUR | Plasminogen activator, urokinase receptor |
| ITGA1 | Integrin, alpha 1 |
| FLOR1 | Folate receptor |
| VCAM1 | Vascular cell adhesion molecule 1 |
| VEGFR3 | Vascular endothelial growth factor receptor 3 |
| CD44 | CD44 molecule |
| SELP | Selectin P |
| CXCR4 | Chemokine (C-X-C motif) receptor 4 |
| CDH5 | Cadherin 5, type 2 (vascular endothelium) |
| CCR5 | Chemokine (C-C motif) receptor 5 |
| PDGFRB | Platelet-derived growth factor receptor, beta polypeptide |
| CD34 | CD34 molecule |
| ITGAL | Integrin, alpha L |
| ITGB2 | Integrin, beta 2 |
| CDH2 | Cadherin 2, type 1, N-cadherin |
| PDGFRA | Platelet-derived growth factor receptor, alpha polypeptide |
| VEGFR1 | Vascular endothelial growth factor receptor 1 |
| VEGFR2 | Vascular endothelial growth factor receptor 2 |
| PECAM1 | Platelet/endothelial cell adhesion molecule 1 |
| c-KIT | Mast/stem cell growth factor receptor |
| PSMA | Prostate-specific membrane antigen |
| THY1 | Thy-1 cell surface antigen |
| TEK | TEK tyrosine kinase, endothelial |
| CCR2 | Chemokine (C-C motif) receptor 2 |
| SELE | Selectin E |
| ENG | Endoglin |
| ITGA6 | Integrin, alpha 6 |
| CDH1 | Cadherin 1, type 1, E-cadherin (epithelial) |
| HER2 | human epidermal growth factor receptor 2 |

### Direct Detection of the ICAM1 Antibody-Antigen Interaction on Live TNBC Cells

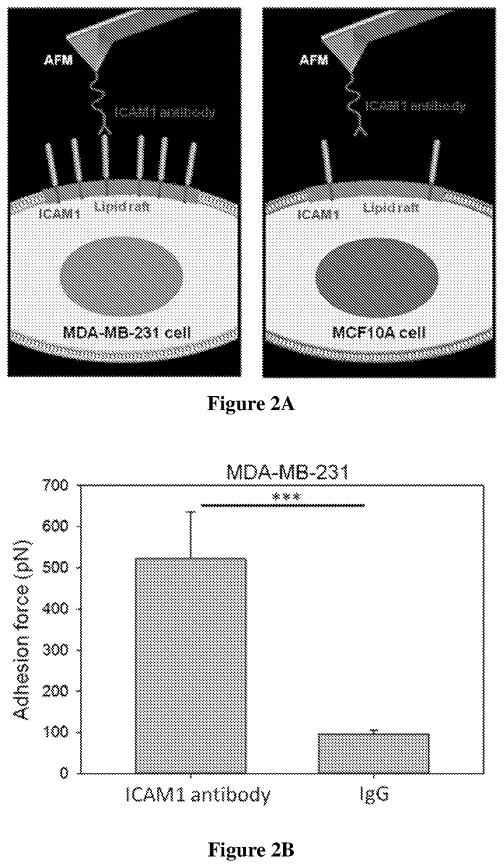

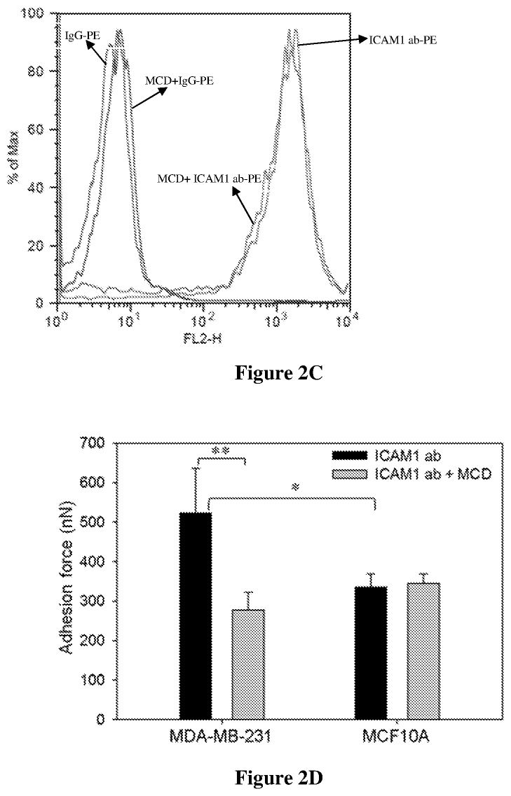

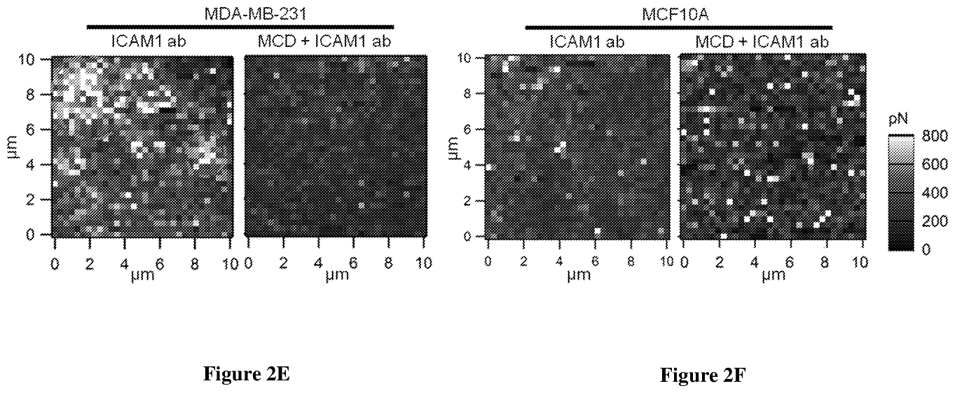

Direct detection of ligand-receptor interactions may hold the key to assessing tumor specificity and for predicting in vivo affinity of targeted therapeutics. In this study, AFM was used to quantitatively probe ICAM1 antigen-antibody interactions on live human TNBC cells (MDA-MB-231) and compared these results with non-neoplastic human mammary epithelial cells (MCF10A). As shown in FIG. 2A, the AFM tip was functionalized with ICAM1 antibodies (1200±300 molecule/μm²), and this functionalized AFM tip-cantilever assembly was used to probe the adhesion forces between the ICAM1 antibody attached on the AFM tip and antigens presented on the cell surface^{10,19,20}. The average adhesion force was quantified from the difference in the approach and retract curves at the pull-off point²¹. As shown in FIG. 2B, the ICAM1 antibody demonstrated an average adhesion force of 523±113 pN on live MDA-MB-231 cells, which was significantly higher than that of its non-targeting counterpart IgG (96±10 pN). The average adhesion forces of the ICAM1 antibody on MDA-MB-231 cells (523±113 pN, black bar) and non-neoplastic MCF10A cells (336±33 pN, black bar) was further compared in FIG. 2D, which indicated that the ICAM1 antibody had a stronger affinity for the MDA-MB-231 cell membrane than the non-neoplastic MCF10A cell membrane. The ICAM1 antibody-antigen binding force on live MDA-MB-231 cells is only 1.6-fold higher than that of non-neoplastic controls (MCF10A cells). It was not expected that the ICAM1 antibody-antigen binding force would be significantly lower than its gene and surface protein overexpression levels (13.9 and 46.4-fold, respectively). However, this in vitro antibody-antigen binding force difference between TNBC and non-neoplastic cells was later found to have a determinative role in regulating both in vitro and in vivo tumor recognition of ICAM1 antibody-directed liposomes, which is more precise and efficient as a predictive factor than established gene and surface protein overexpression levels. From these results, the TNBC tumor-recognition affinity of ICAM1 antibody as 187 pN was calculated with the following equation:

Tumor-recognition Affinity_{TNBC}=Adhesion Force_{TNBC}−Adhesion Force_{Non-neoplastic}(pN)

In addition to the overexpression level, the organization of antigens on the cell membrane is another key factor driving differences in antibody-antigen binding behavior¹¹. AFM-detected adhesive events were also used to spatially map the binding forces on MDA-MB-231 (FIG. 2E) and MCF10A (FIG. 2F) cell surface. As shown in FIG. 2E, this AFM map reveals that ICAM1 molecules were heterogeneously organized on the cell surface and adhesion force “hot-spots” for the ICAM1 antibody were observed on MDA-MB-231 cell membranes (highlighted in FIG. 2E). These “hot-spots” are predicted to be the primary binding sites for ICAM1 antibody-directed nanomedicine due to the high binding forces. ICAM1 molecules may be enriched in membrane lipid rafts of MDA-MB-231 cells to facilitate functional signaling²². Lipid rafts, present in cell membranes, are gel-phase domains rich in cholesterol and cell membrane proteins that affect antibody-antigen interactions in a cholesterol-dependent manner^{23,24}. In order to determine whether ICAM1 adhesion forces are dependent on the organization of ICAM1 molecules in lipid rafts on the cell membrane, both MDA-MB-231 and MCF10A cells were treated with methyl-beta-cyclodextrin (MCD), a cholesterol-depleting drug that disrupts lipid rafts, and then the average binding force was re-measured. As shown in FIG. 2C, MCD treatment did not affect the average ICAM1 expression on MDA-MB-231 cell surface. Similar results were also observed in other cell types¹⁵. While MCD treatment had no obvious effect on ICAM1 cell surface expression, MCD did impede the ICAM1 antibody-antigen interaction by delocalizing cell membrane lipid raft-associated molecules²⁵. In FIG. 2E, the “hot spots” of ICAM1 adhesion events disappeared after MCD treatment, and the average adhesion force between the ICAM1 antibody and MDA-MB-231 cells significantly decreased from 523±113 pN to 277±46 pN in the presence of MCD (FIG. 2D, grey bars) correlating with disperse adhesion maps (FIGS. 2E and 2F). No difference was observed between MCF10A cells treated with or without MCD due to its ICAM1 deficiency (FIGS. 2D and 2F). Therefore, the selective and strong ICAM1 antibody binding force with the MDA-MB-231 cell membrane is attributed to both the overexpression and the organization of ICAM1 molecules presented on MDA-MB-231 cell membranes.

### Construction of ICAM1 Antibody-Directed Liposomes

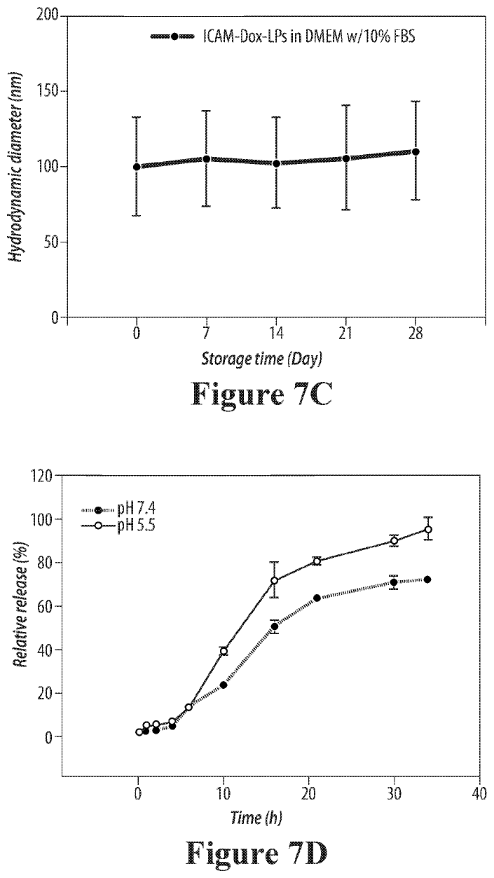

Next, a series of ICAM1 antibody-directed liposomes were engineered to investigate the implications of the in vitro ICAM1 antibody-antigen binding force in TNBC tumor recognition. ICAM1 antibody-conjugated, doxorubicin-encapsulating liposomes (ICAM-Dox-LPs) were prepared as a TNBC-specific therapeutic agent, as described in FIG. 7A. ICAM-Dox-LPs were comprised of 95 mol % DOPC and 5 mol % DSPE-PEG-COOH. The PEG chain (2 kDa) in DSPE-PEG-COOH improves liposome circulation time^{26,27}. ICAM1 antibody or non-specific immunoglobulin G (IgG) was conjugated to the carboxyl terminus of the PEG chain. IgG-conjugated, Dox encapsulating liposomes (IgG-Dox-LPs) were prepared as controls. As-synthesized liposomes are characterized in Table 2. Hydrodynamic diameters of ICAM-Dox-LPs and IgG-Dox-LPs were 105±31 and 101±24 nm, respectively, as determined by dynamic light scattering (DLS, FIG. 7B). Polydispersity indexes (PDIs) of both liposomes were close to 0.1, demonstrating uniformity. In addition, the zeta potentials of ICAM-Dox-LPs and IgG-Dox-LPs were −8.8±6.7 and −4.8±4.1 mV, respectively. The transmembrane gradient method was used to encapsulate Dox in liposomes²⁸. The Dox encapsulation efficiencies of ICAM-Dox-LPs (92.0±1.6%) and IgG-Dox-LPs (91.5±0.5%) were comparable. Furthermore, the surface densities of conjugated ICAM1 antibody or non-specific IgG were quantified as 3,040±20 molecules/μm² for ICAM-Dox-LPs and 3,100±28 molecules/μm² for IgG-Dox-LPs. This is equivalent to approximately 96 molecules per liposome. The storage stability of constructed ICAM-Dox-LPs was also investigated and it was found that it maintained its hydrodynamic size in DMEM with 10% FBS for 28 days without aggregation (FIG. 7C). The release profiles of Dox from ICAM-Dox-LPs at pH 7.4 and 5.5 were measured in order to mimic the extra- and intra-cellular environments, respectively (FIG. 7D)²⁹⁻³¹ and found ICAM-Dox-LP released its cargo faster at the lower pH.

TABLE 2
Characterization of as-synthesized ICAM-Dox-LP and IgG-Dox-LP.

| | Size | Polydispersity | Zeta potential | Encapsulation Ratio | Antibody density |
|---|---|---|---|---|---|
| Sample | (nm) | index | (mV) | (%) | (molecules/μm²) |
| ICAM-Dox-LP | 105 ± 31 | 0.113 | −8.8 ± 6.7 | 92.0 ± 1.6 | 3,040 ± 20 |
| IgG-Dox-LP | 101 ± 24 | 0.071 | −4.8 ± 4.1 | 91.5 ± 0.5 | 3,100 ± 28 |

### In Vitro Binding Affinity of ICAM1 Antibody-Directed Liposomes

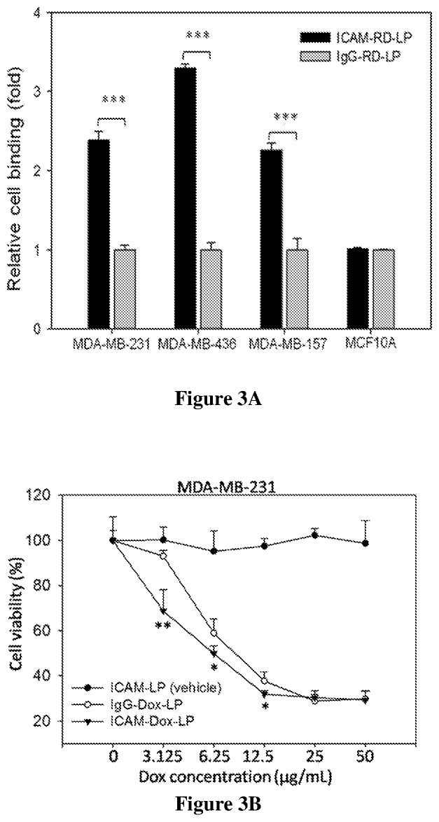

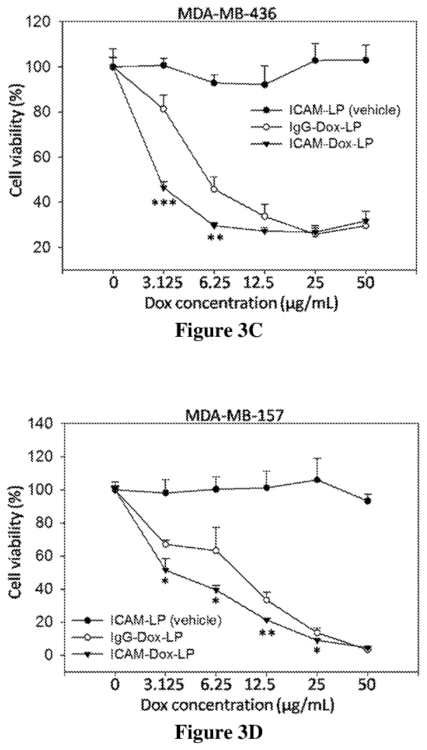

First, the in vitro TNBC cell binding of ICAM1-directed liposomes was quantified by flow cytometry. Liposomes encapsulating rhodamine-dextran (RD, 10 kDa) were used to avoid the cytotoxic effect of doxorubicin. Cellular binding and uptake of the ICAM1 antibody or IgG labeled, RD encapsulating liposomes (ICAM-RD-LPs or IgG-RD-LPs) were assessed on three TNBC cell lines: MDA-MB-231, MDA-MB-436 and MDA-MB-157, in comparison with non-neoplastic MCF10A cells. As shown in FIG. 3A, MDA-MB-231, MDA-MB-436 and MDA-MB-157 cells demonstrated 2.4, 3.3 and 2.3-fold higher binding of ICAM-RD-LPs diluted in cell culture medium containing 10% serum relative to non-specific IgG-RD-LPs, respectively. No difference in binding and uptake between ICAM-RD-LPs and IgG-RD-LPs was detected on MCF10A cells due to its lack of ICAM1 expression. These findings demonstrate that ICAM1 antibodies covalently conjugated on the surface of ICAM-RD-LPs maintain their activity and selectively recognize TNBC cells via the ICAM1 antibody-antigen interaction. The in vitro TNBC-liposome binding is consistent with the high binding forces measured on TNBC cells relative to MCF10A cells (FIGS. 2D and 2F).

To assess the TNBC-specific cytotoxicity of ICAM-Dox-LPs, proliferation assays were performed on the three TNBC cell lines treated with ICAM-Dox-LPs as a function of Dox concentration. ICAM-LPs, Free Dox, and IgG-Dox-LPs were selected as controls. In all three TNBC cell lines, ICAM-Dox-LPs demonstrated substantially higher in vitro cytotoxicity in comparison to non-specific IgG-Dox-LPs. Half maximal inhibitory concentrations (IC50s) were calculated from the cytotoxicity curves. For MDA-MB-231 cells (FIG. 3B), the IC50s were 6.5 μg/mL for ICAM-Dox-LPs, 11.4 μg/mL for IgG-Dox-LPs, and 7.4 μg/mL for free Dox. Similar trends were observed in the MDA-MB-436 and MDA-MB-157 TNBC cell lines (FIGS. 3C and 3D). ICAM-LPs did not exhibit cytotoxicity in TNBC cells. These findings demonstrate that introducing a TNBC specific-binding function to liposomal doxorubicin via the ICAM1 antibody can significantly improve its cytotoxicity to TNBC cells relative to non-specific IgG-Dox-LPs.

### In Vivo Tumor Recognition and Efficacy of ICAM1 Antibody-Directed Liposomes

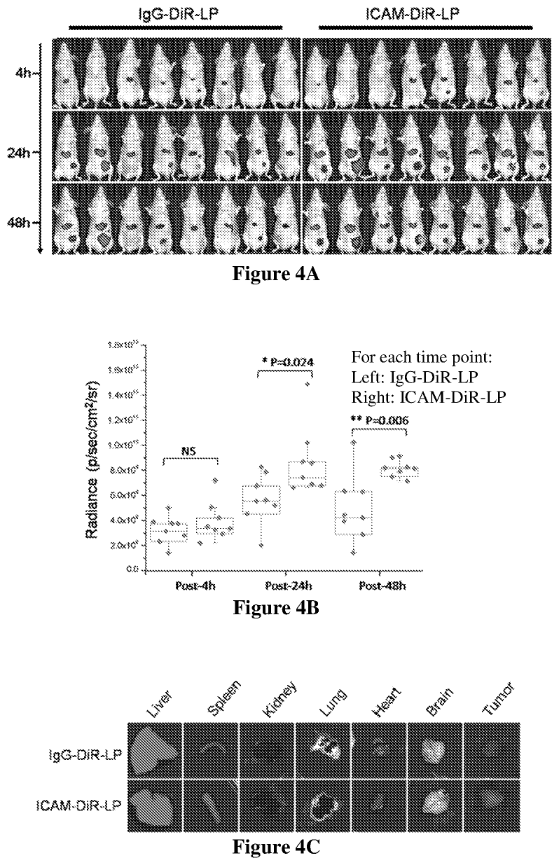

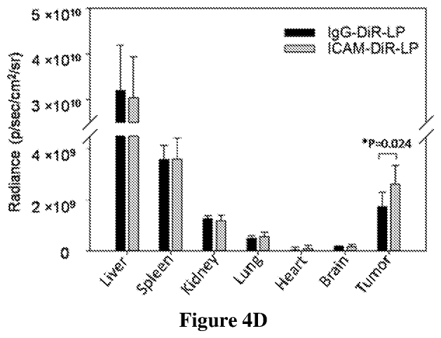

To determine whether increased ICAM-Dox-LP affinity for TNBC cells translates into improved liposome accumulation in TNBC tumors in vivo, the distribution of ICAM1 antibody-directed liposomes was examined by near-infrared (NIR) fluorescent imaging in a mouse breast cancer model. MDA-MB-231 cells were orthotopically implanted in immunodeficient nude mice. NIR fluorescent imaging was performed on two groups of tumor-bearing mice injected with either ICAM1 antibody or IgG conjugated liposomes labeled with a NIR dye DiR (ICAM-DiR-LPs or IgG-DiR-LPs). Each group was scanned at 4, 24, and 48 hours post injection. The representative images in FIG. 4A show that accumulation of ICAM-DiR-LPs was significantly increased at TNBC tumor sites relative to that of non-specific IgG-DiR-LPs. Mice injected with ICAM-DiR-LPs exhibited a 1.2-fold (4 hours), a 1.5-fold (24 hours), and a 1.6-fold (48 hours) increase in tumor-specific fluorescence compared to those injected with IgG-DiR-LPs, suggesting that ICAM-DiR-LPs significantly improved TNBC tumor accumulation by actively targeting the TNBC tumor via ICAM1 antibody-antigen interaction (FIG. 4B).

The biodistribution of ICAM1 antibody-directed liposomes was evaluated by quantifying ex vivo NIR fluorescent signals in collected organs and tumors. FIGS. 4C and 4D show comparative liposome accumulation in six organs (liver, spleen, lung, kidney, brain, and heart) and TNBC tumors harvested from mice at 48 hours after a single tail vein administration of IgG-DiR-LPs or ICAM-DiR-LPs. Correlating with the in vivo imaging results, the accumulation of ICAM-DiR-LPs in TNBC tumors was approximately 1.5-fold higher than that of IgG-DiR-LPs. For the six organs analyzed, liver and spleen were the two primary accumulation sites for both ICAM1-targeted and non-specific-IgG liposomes, as observed in other liposome studies^{32,33} and there was no significant difference observed between ICAM-DiR-LP and IgG-DiR-LP groups. It is noteworthy that the in vivo and ex vivo MDA-MB-231 tumor accumulation of ICAM-DiR-LPs (1.6 and 1.5-fold over IgG-DiR-LP) are precisely consistent with the in vitro ICAM1 antibody-antigen binding force on live MDA-MB-231 cells (1.6-fold over non-neoplastic controls), but not with ICAM1 mRNA and surface protein overexpression levels (13.9 and 46.4-fold over non-neoplastic controls) due to the determinative role of antibody-antigen interaction in tumor recognition.

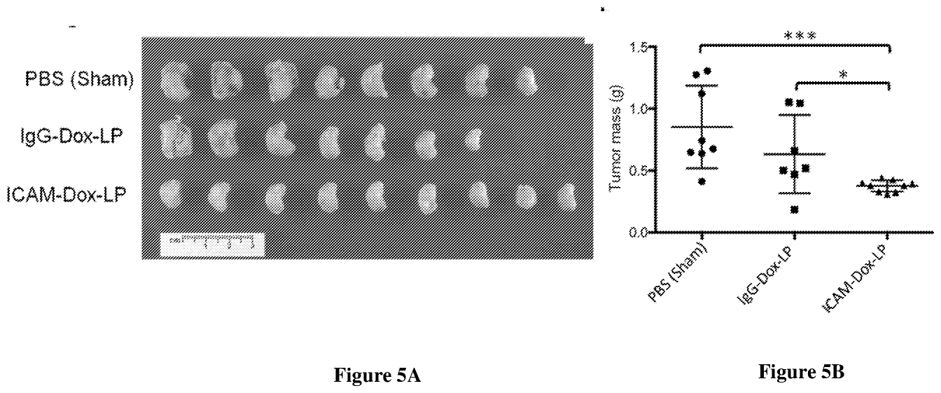

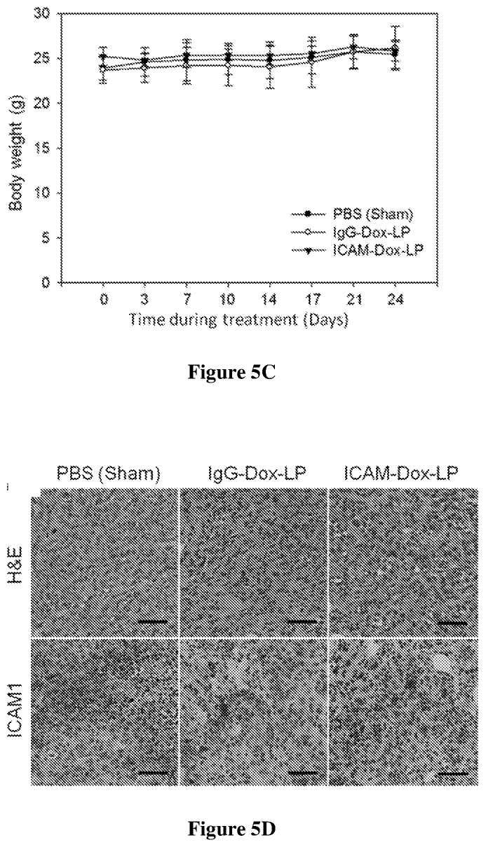

It was further examined whether ICAM1 antibody-directed liposomes were able to convert their in vivo TNBC tumor-targeting activity into improved therapeutic efficacy. ICAM-Dox-LPs were injected intravenously into nude mice bearing orthotopic TNBC tumors (MDA-MB-231 cells). PBS and non-targeted IgG-Dox-LPs were also tested as controls. After a 24-day treatment regimen, the administration of ICAM-Dox-LPs efficiently inhibited TNBC tumor growth in comparison with PBS and IgG-Dox-LP groups (FIG. 5A). Quantified tumor mass results (FIG. 5B) further revealed that ICAM-Dox-LPs significantly inhibited TNBC tumor growth by at least 41% relative to control groups (PBS and IgG-Dox-LPs), equivalent to an approximately 1.7-fold increased therapeutic efficacy over IgG-Dox-LP that closely matches the in vitro ICAM1 antibody-antigen binding force (1.6-fold) and in vivo tumor recognition (1.6-fold). All groups of mice maintained their body weight without significant loss during these treatment periods (FIG. 5C). Hematoxylin and eosin (H&E) staining and immunohistochemical staining of ICAM1 were performed on sections of excised TNBC tumors (FIG. 5D). High expression levels of ICAM1 were present in TNBC tumors from all three treatment groups (PBS, IgG-Dox-LP, and ICAM-Dox-LP), indicating that the differences in the therapeutic efficacy among the three treatment groups was not due to any difference in ICAM1 levels in tumors. In summary, the results herein confirm that ICAM-Dox-LPs rely on their ICAM1 antibody-mediated binding force to specifically recognize ICAM1 overexpressing tumors in vivo and inhibit tumor growth.

### DISCUSSION

Efficient tumor-specific delivery of therapeutics in vivo remains a challenge in nanomedicine research. Herein, a novel strategy, utilizing AFM, which predicts in vivo tumor recognition of antibody-directed nanomedicines, is reported. The antibody-antigen interaction of cancer targets was directly measured using antibody-functionalized AFM on live TNBC cells in comparison with non-neoplastic human mammary epithelial cells. This method was used to develop a simple and potentially universal metric for predicting the in vivo tumor recognition capacity of nanomedicine (FIG. 6). This approach can also be used as a metric for evaluating tumor-recognition efficiency of receptor-mediated nanomedicines. Compared with other established predictive factors (e.g. gene or protein overexpression levels), the proof-of-principle animal studies showed that this AFM-based method provides a more precise and efficient evaluation of antibody-antigen interaction-based tumor-binding events for antibody-directed liposomes. Furthermore, the high-resolution imaging feature of AFM enables the spatio-temporal visualization of specific binding sites on live TNBC cell surfaces, providing information on the localization and organization of cell membrane antigen that is critical for antibody-antigen interactions.

The findings presented herein show that the in vitro antibody-antigen binding force of ICAM1 correlates with the in vivo TNBC tumor accumulation of ICAM1 antibody-directed liposomes may have direct implications for the design of TNBC-targeted therapeutics.^{10,11,19,20,34,35} Li et al. reported that Rituximab, a FDA-approved CD20 antibody for Non-Hodgkin's Lymphoma (NHL) treatment, exhibited a binding force of 54±34 pN on patient-derived NHL B cells and 21±19 pN on normal red blood cells, indicating a specific NHL tumor recognition affinity of 33 pN.³⁴ In comparison, the TNBC tumor-recognition affinity of ICAM1 antibody was quantified as being 187 pN, which is 5.6-fold higher than the NHL tumor recognition affinity of Rituximab. It was reasoned that combining a TNBC-specific ligand e.g., ICAM1 antibody, to clinically-approved nanomedicines (e.g. Doxil or Abraxane), would enable it to more efficiently recognize and target TNBC tumors and metastatic lesions and, in turn, may increase the drug dosage in tumors, reduce non-specific uptake and attenuate adverse side-effects. The in vivo biodistribution studies using an orthotopic mouse TNBC model validated that the ICAM1 antibody-directed liposomes achieved approximately 80% more accumulation in tumor sites than the non-targeted IgG controls.

In summary, it was demonstrated herein that the antibody-antigen binding force data on live cancer cells, acquired through AFM, may be used as a novel metric to predict in vivo tumor recognition of antibody-directed nanomedicines. This AFM method used biomechanic parameters that can be measured on individual cells and is, in principle, applicable to a broad range of tumor-targeting molecules (e.g. natural ligands, engineered peptides or aptamers). Moreover, the application of this methodology in the screening and identification of novel molecular targets may also be extended to multiple cancers.

### Materials and Methods

### Materials

Dulbecco's phosphate buffered saline (PBS), 0.25% trypsin/2.6 mM ethylenediaminetetraacetic acid (EDTA) solution, GIBCO® Dulbecco's Modified Eagle Medium (DMEM), and GIBCO®DMEM/F12 (1:1) were purchased from INVITROGEN™ (Carlsbad, Calif., USA). Quantum Simply Cellular microbeads were purchased from Bangs Laboratory (Fishers, Ind., USA). For Phycoerythrin (PE)-conjugated antibodies used in flow cytometric analysis, PE-conjugated mouse anti human VEGFR1 antibody, PE-conjugated mouse anti human VEGFR2 antibody, and PE-conjugated FLOR1 antibody were purchased from R&D Systems (Minneapolis, Minn., USA). All other PE-conjugated antibodies and immunoglobulin G (IgG) isotype controls for FACS measurements were purchased from BIOLEGEND® (San Diego, Calif., USA). 1-Ethyl-3-(3-dimethylaminopropyl) carbodiimide hydrochloride (EDC), N-hydroxysuccinimide (NHS), bovine serum albumin (BSA), ammonium sulfate, anhydrous dimethyl sulfoxide (DMSO), doxorubicin, and Nanosep 300k Omega centrifugal device were purchased from SIGMA-ALDRICH™ (St. Louis, Mo., USA). Corning Costar Transwell Permeable Supports and Lab-Tek II Chamber Slide System and lipophilic carbocyanine DiOC18 (7) (DiR) were purchased from THERMO FISHER™ Scientific (Waltham, Mass., USA). 1,2-dioleoyl-sn-glycero-3-phosphocholine (DOPC) and 1,2-distearoyl-sn-glycero-3-phosphoethanolamine-N-[carboxy(polyethylene glycol)-2000] (DSPE-PEG-COOH) were purchased from Avanti Polar Lipids (Alabaster, Ala., USA). Human breast cancer tissue microarray (BR1505B) was purchased from US Biomax (Rockville, Md., USA). Fluorogel with tris buffer was purchased from Electron Microscopy Sciences (Hatfield, Pa., USA). RNeasy mini kit was purchased from QIAGEN™ (Valencia, Calif., USA). Fetal bovine serum was purchased from Atlanta Biologicals (Flowery Branch, Ga., USA).

### Cell Culture

Three human TNBC cell lines (MDA-MB-231, MDA-MB-436, and MDA-MB-157) and non-neoplastic MCF10A cells were obtained from American Type Culture Collection (ATCC, Manassas, Va.) and cultured in DMEM or DMEM/F12 (1:1) medium with supplements, respectively. All cells were cultured in a 37° C. humidified incubator with 5% CO₂.

### Flow Cytometric Analysis

10⁶ cells were collected and rinsed twice through suspension-spin cycles. Cells were incubated with 1% BSA in PBS for 30 min in an ice bath. After BSA blockage, cells were incubated with fluorophore-conjugated antibody for 1 hour at room temperature. Cells were rinsed with 1% BSA in PBS three times, resuspended in PBS, and evaluated by a BD FACSCalibur flow cytometer (BD Biosciences). Quantification of cell surface antigen was determined with reference to Quantum Simply Cellular microbeads, using the protocol provided by the manufacturer.

### Atomic Force Microscopy (AFM)

AFM was used to obtain the spatial organization and affinity of ICAM1 on live human TNBC MDA-MB-231 or non-neoplastic MCF10A cells as previously reported.¹¹ Briefly, AFM experiments were performed with an Asylum MFP-3D SA AFM (Asylum Research, CA), with silicon nitride, four sided pyramid tips (BL-TR400PB-35, Asylum Research, CA). The spring constant of tips were properly calibrated every time by the Thermal Method, and all tips which have been used had a spring constant between 0.02 to 0.04 N/m. The cells were cultured in a 35 mm Petri dish which was placed under an AFM head; AFM worked on contact mode and the trigger voltage was 0.5 V. The scan rate was 1 Hz, and scan size was 10 μm×10 μm. The antibody-antigen binding force was calculated from the force-distance curve, and five cells were measured for each sample. In order to minimize the error of the measurements, one series of experiments have been performed with the same AFM tip at the same experimental condition.

### Preparation of ICAM1 Antibody-Directed Liposomes

ICAM1 antibody-conjugated, doxorubicin-encapsulating liposomes (ICAM-Dox-LPs) were prepared by the extrusion method as previously described³⁶⁻³⁹. A mixture of DOPC:DSPE-PEG-COOH (95:5, mol:mol) was solubilized in chloroform and dried in a rotary evaporator under reduced pressure at room temperature. The lipid film was dissolved in 1 mL DMSO:EtOH (7:3, v:v). The lipid solution was injected in 9 mL 240 mM (NH₄)₂SO₄ buffer (pH 5.4) while being agitated at 650 rpm with a stir bar to yield a 5 mM lipid solution. Liposomes were extruded via a Northern Lipids Extruder with a 100 nm polycarbonate nanoporous membrane. After extrusion, the liposome solution was dialyzed in phosphate buffered saline (PBS pH 7.4) using a Slide-A-Lyzer dialysis cassette (MWCO 20 kDa) overnight at room temperature (RT). Dox was encapsulated in the liposomes via an active transmembrane pH gradient method. Liposomes were incubated within a Dox solution (1 mg/mL in PBS) for 6 hours to allow Dox loading. Obtained Dox-loaded liposomes were dialyzed in PBS (pH 7.4) using a Slide-A-Lyzer dialysis cassette (MWCO 20 kDa) for 12 hours at RT to remove excess Dox. Liposomes were conjugated to ICAM1 antibody via the DSPE-PEG-COOH anchor. EDC (2 mg) and NHS (3 mg) were mixed with 1 mmol of lipid (liposomes) in PBS (pH 7.4) and incubated for 6 hours at RT. A Slide-A-Lyzer dialysis cassette (MWCO 10 kDa) was used to remove unreacted EDC and NHS. ICAM1 antibody or the IgG isotype was then added to EDC-modified liposomes at a molar ratio of 1:1000 (antibody:phospholipid) and incubated overnight at RT. Unreacted antibodies were removed by dialysis using a Float-A-Lyzer G2 (MWCO 1,000 KD) dialysis device. In liposome binding experiments, ICAM1 antibody-conjugated, rhodamine-dextran encapsulating liposomes (ICAM-RD-LPs) were prepared and tested. For ICAM-RD-LPs, the preparation process was similar to that of the ICAM-Dox-LPs except that the 1 mL lipid solution was added to a 9 mL rhodamine-dextran solution (1 mg/mL). In IVIS near-infrared (NIR) fluorescent imaging experiments, ICAM1 antibody or IgG-conjugated, DiR-labeled liposomes (ICAM-DiR-LP or IgG-DiR-LP) were prepared using a similar procedure except adding 0.2 mol % DiR, a NIR fluorescent lipid dye to lipid mixture solution. No Dox was encapsulated in either ICAM-DiR-LP or IgG-DiR-LP.

The antibody density conjugated on liposomes was quantified. Liposomes cannot be detected by flow cytometry because of their size. Therefore, 2 μm borosilicate beads were encapsulated within DOPC:DSPE-PEG-COOH (95:5, mol:mol) liposomes by sonicating small unilamellar liposomes with microbeads in PBS for 6 hours. Microbeads were rinsed three times in PBS via suspension-spin cycles to separate free liposomes. Conjugation of PE-ICAM1 antibody or PE-IgG (nonspecific binding) to microbeads encapsulating liposomes was performed using EDC/NHS chemistry. The surface density of ICAM1 antibody conjugated to each microbead was determined with reference to Quantum Simply Cellular microbeads, which have defined numbers of antibody binding sites per bead. Liposome size and zeta potential were measured by dynamic light scattering on a Zeta-PALS analyzer (Brookhaven Instruments, Holtsville, N.Y.) in PBS (pH 7.4).

### Sustained Release Profile of ICAM1 Antibody-Directed Liposomes

Release of Dox from ICAM-Dox-LPs was carried out in PBS at pH 5.5 and 7.4. The ICAM-Dox-LP solution (1 mL, 200 μg/mL) was added to a dialysis tube (MWCO 12.4 kDa). The dialysis tube was placed in a beaker with 50 mL PBS (pH 5.5 or 7.4). Then the beaker was sealed with parafilm and incubated at 37° C. on a shaker (100 rpm). For each time point, three 100 μL samples were collected from the solution outside of dialysis tube and the fluorescence intensity was measured on a SpectraMaxGEMIN XPS fluorescence spectrophotometer (Molecular Devices Corp, Sunnyvale, Calif., USA). The Dox excitation and emission wavelengths were 485 nm and 590 nm, respectively. The release rate of Dox was calculated based on a standard fluorescence concentration calibration curve.

### In Vitro Cellular Binding Assay

Quantitative analysis of liposome binding to TNBC cells (MDA-MB-231, MDA-MB-436, MDA-MB-157, and MCF10A (control)) was conducted with flow cytometry. Cells were seeded in 6-well plates (3×10⁵ cells/well) and allowed to adhere overnight. The attached cells were incubated for 4 hours at 37° C. with (1) rhodamine-dextran encapsulated nonspecific (IgG) liposomes (IgG-RD-LPs) and (2) ICAM-RD-LPs. The concentration used was 1 μmol lipid/10⁶ cells. All liposome treated cells were washed with PBS, harvested using a 0.25% trypsin/2.6 mM EDTA solution, and washed with PBS (pH 7.4) three times. Binding data were acquired using a BD FACSCalibur flow cytometer and analyzed using FLOWJO® software. The binding fold-over non-specific IgG-RD-LPs was calculated by dividing the mean fluorescence intensity for ICAM-RD-LP stained cells by that of the IgG-RD-LPs.

### In Vitro Cytotoxicity Assays

In vitro cytotoxicity of ICAM-Dox-LPs on TNBC cells was evaluated using a cell viability assay. Five thousand cells (MDA-MB-231, MDA-MB-436, and MDA-MB-157) were seeded in each well of a 96 well plate and incubated for 24 hours. Cells were treated with (1) ICAM-LP without Dox; (2) Free Dox; (3) non-specific IgG-Dox-LPs and (4) ICAM-Dox-LPs for 4 hours. Cells were rinsed twice with PBS and grown for 48 hours. Cell viability was determined by a Dojindo cell counting kit using the manufacturer's protocol (Rockville, Md.).

### Orthotopic TNBC Mouse Model and Treatments

In vivo studies were performed according to the protocols approved by the Institutional Animal Care and Use Committees of The City College of New York and Boston Children's Hospital. Breast tumors were orthotopically implanted by injecting 5×10⁶ MDA-MB-231 cells into the fourth mammary fat pad of female nude mice (Charles River). Mice were randomized into various treatment groups (n=8-10 for each group). For the in vivo fluorescent imaging experiments, tumors were allowed to develop for 2-3 weeks until they were at least 200 mm³ in volume. In vivo fluorescent imaging was performed on the tumor-bearing mice that were injected intravenously with IgG-DiR-LP or ICAM-DiR-LP (at dosage of 20 mg lipids/kg mouse weight) using tail-vein injection. At 4, 24, and 48 hours after the injection, in vivo fluorescence imaging was performed using an IVIS Lumina II (Caliper, Hopkington, Mass.). At 48 hours post injection, the mice were sacrificed and the ex vivo fluorescence intensity of various organs (brain, heart, liver, lung, kidney and spleen) and tumor was measured using an IVIS Lumina II System.

For in vivo therapeutic efficacy experiments, tumors were allowed to develop for 1-2 weeks until they reached 100 mm³ in volume. Each group of mice was then treated with PBS (sham), IgG-Dox-LP, or ICAM-Dox-LP (2.5 mg/kg per dosage, twice a week). All injections for treatments were performed intravenously via retro-orbital injection in 50 μL PBS. Twenty-four days after treatment, orthotopic tumors were excised to measure their mass. H&E staining and immunohistochemical staining of ICAM1 were performed on excised MDA-MB-231 tumor slides using standard protocols as previously described¹².

### Statistical Analysis

All of the experimental data were obtained in triplicate unless otherwise mentioned and are presented as mean±standard deviation. Statistical comparison by analysis of variance was performed at a significance level of p<0.05 based on a Student's t-test.

### REFERENCES

1. Bertrand, N.; Wu, J.; Xu, X.; Kamaly, N.; Farokhzad, 0. C. Adv. Drug Deliv. Rev. 2014, 66, 2-25.
2. Wicki, A.; Witzigmann, D.; Balasubramanian, V.; Huwyler, J. J. Controlled Release 2015, 200, 138-157.
3. Xu, X.; Ho, W.; Zhang, X.; Bertrand, N.; Farokhzad, O. Trends Mol. Med. 2015, 21 (4), 223-232.
4. LoRusso, P.; Krop, I.; Miller, K.; Ma, C.; Siegel, B. A.; Shields, A. F.; Molnar, I.; Wickham, T.; Reynolds, J.; Campbell, K.; Hendriks, B.; McClure, T.; Moyo, V.; Munster, P. Cancer Res. 2015, 75 (15 Supplement), CT234-CT234.
5. Roy, R.; Rodig, S.; Bielenberg, D.; Zurakowski, D.; Moses, M. A. J. Biol. Chem. 2011, 286 (23), 20758-20768.
6. Roy, R.; Wewer, U. M.; Zurakowski, D.; Pories, S. E.; Moses, M. A. J. Biol. Chem. 2004, 279 (49), 51323-51330.
7. Roy, R.; Moses, M. A. Breast Cancer Res. Treat. 2012, 131 (3), 731-741.
8. Roy, R.; Yang, J.; Moses, M. A. J. Clin. Oncol. 2009, 27 (31), 5287-5297.
9. Yang, J.; Bielenberg, D. R.; Rodig, S. J.; Doiron, R.; Clifton, M. C.; Kung, A. L.; Strong, R. K.; Zurakowski, D.; Moses, M. A. Proc. Natl. Acad. Sci. 2009, 106 (10), 3913-3918.
10. Fernandez, C. A.; Roy, R.; Lee, S.; Yang, J.; Panigrahy, D.; Van Vliet, K. J.; Moses, M. A. J. Biol. Chem. 2010, 285 (53), 41886-41895.
11. Wang, B.; Guo, P.; Auguste, D. T. Biomaterials 2015, 57, 161-168.
12. Guo, P. †; Huang, J. †; Wang, L.; Jia, D.; Yang, J.; Dillon, D. A.; Zurakowski, D.; Mao, H.; Moses, M. A.; Auguste, D. T. Proc. Natl. Acad. Sci. 2014, 111 (41), 14710-14715. †contributed equally.
13. Guo, P. †; Yang, J. †; Jia, D.; Moses, M. A.‡; Auguste, D. T. ‡ Theranostics 2016, 6 (1), 1-13. †contributed equally. ‡contributed equally.
14. Yook, S.; Cai, Z.; Lu, Y.; Winnik, M. A.; Pignol, J.-P.; Reilly, R. M. Mol. Pharm. 2015, 12 (11), 3963-3972.
15. Yang, L.; Sajja, H. K.; Cao, Z.; Qian, W.; Bender, L.; Marcus, A. I.; Lipowska, M.; Wood, W. C.; Wang, Y. A. Theranostics 2014, 4 (1), 106-118.
16. Devulapally, R.; Sekar, N. M.; Sekar, T. V.; Foygel, K.; Massoud, T. F.; Willmann, J. K.; Paulmurugan, R. ACS Nano 2015, 9 (3), 2290-2302.
17. Deng, Z. J.; Morton, S. W.; Ben-Akiva, E.; Dreaden, E. C.; Shopsowitz, K. E.; Hammond, P. T. ACS Nano 2013, 7 (11), 9571-9584.
18. Mendes, T. F. S.; Kluskens, L. D.; Rodrigues, L. R. Adv. Sci. 2015, n/a-n/a.
19. Lee, S.; Mandic, J.; Van Vliet, K. J. Proc. Natl. Acad. Sci. 2007, 104 (23), 9609-9614.
20. Van Vliet, K. J.; Hinterdorfer, P. Nano Today 2006, 1 (3), 18-25.
21. Hinterdorfer, P.; Baumgartner, W.; Gruber, H. J.; Schilcher, K.; Schindler, H. Proc. Natl. Acad. Sci. 1996, 93 (8), 3477-3481.
22. Tilghman, R. W.; Hoover, R. L. FEBS Lett. 2002, 525 (1-3), 83-87.
23. Silvius, J. R. Biochim. Biophys. Acta BBA—Biomembr. 2003, 1610 (2), 174-183.
24. Simons, K.; Toomre, D. Nat. Rev. Mol. Cell Biol. 2000, 1 (1), 31-39.
25. Gunawan, R. C.; Auguste, D. T. Mol. Pharm. 2010, 7 (5), 1569-1575.
26. Photos, P. J.; Bacakova, L.; Discher, B.; Bates, F. S.; Discher, D. E. J. Controlled

Release 2003, 90 (3), 323-334.
27. Harris, J. M.; Chess, R. B. Nat. Rev. Drug Discov. 2003, 2 (3), 214-221.
28. Bolotin, E. M.; Cohen, R.; Bar, L. K.; Emanuel, N.; Ninio, S.; Barenholz, Y.; Lasic, D.

D. J. Liposome Res. 1994, 4 (1), 455-479.
29. You, J.-O.; Auguste, D. T. Nano Lett. 2009, 9 (12), 4467-4473.
30. You, J.-O.; Auguste, D. T. Biomaterials 2010, 31 (26), 6859-6866.
31. You, J.-O.; Almeda, D.; Ye, G. J.; Auguste, D. T. J. Biol. Eng. 2010, 4, 15.
32. Lee, J. S.; Ankone, M.; Pieters, E.; Schiffelers, R. M.; Hennink, W. E.; Feijen, J. J.

Controlled Release 2011, 155 (2), 282-288.
33. Paolino, D.; Cosco, D.; Racanicchi, L.; Trapasso, E.; Celia, C.; lannone, M.; Puxeddu, E.; Costante, G.; Filetti, S.; Russo, D.; Fresta, M. J. Controlled Release 2010, 144 (2), 144-150.
34. Li, M.; Xiao, X.; Liu, L.; Xi, N.; Wang, Y.; Dong, Z.; Zhang, W. Exp. Cell Res. 2013, 319 (18), 2812-2821.
35. Shi, X.; Xu, L.; Yu, J.; Fang, X. Exp. Cell Res. 2009, 315 (16), 2847-2855.
36. Gunawan, R. C.; Auguste, D. T. Biomaterials 2010, 31 (5), 900-907.
37. Gunawan, R. C.; Almeda, D.; Auguste, D. T. Biomaterials 2011, 32 (36), 9848-9853.
38. Guo, P.; You, J.-O.; Yang, J.; Moses, M. A.; Auguste, D. T. Biomaterials 2012, 33 (32), 8104-8110.
39. Guo, P.; You, J.-O.; Yang, J.; Jia, D.; Moses, M. A.; Auguste, D. T. Mol. Pharm. 2014, 11 (3), 755-765.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of identifying a cell surface target, the method comprising:
(i) contacting a cell with an atomic force microscopy (AFM) probe functionalized with a ligand that associates with a cell surface molecule of the cell;
(ii) dissociating the AFM probe from the cell surface molecule;
(iii) measuring an adhesion force between the ligand and the cell surface molecule; and
(iv) identifying the cell surface molecule as a cell surface target.

2. The method of claim 1, wherein the cell is a cancer cell.

3. The method of claim 2, wherein the cancer cell is a breast cancer cell.

4. The method of claim 3, wherein the breast cancer cell is a triple negative breast cancer cell (TNBC).

5. The method of any one of claims 1-4, wherein the cell surface molecule is a protein, a lipid, or a carbohydrate.

6. The method of any one of claims 1-5, wherein the cell surface molecule is Intercellular Adhesion Molecule 1 (ICAM1).

7. The method of any one of claims 1-6, wherein the ligand is selected from the group consisting of: antibodies, antibody fragments, synthetic peptides, natural ligands, aptamers, small molecules, and live cells.

8. The method of claim 6 or claim 7, wherein the ligand is an ICAM1 antibody.

9. The method of any one of claims 1-8, wherein the ligand is covalently conjugated to the AFM probe.

10. The method of any one of claims 1-9, wherein the cell is a live cell.

11. The method of any one of claims 1-10, wherein the method is carried out in vitro.

12. The method of any one of claims 1-10, wherein the method is carried out ex vivo.

13. The method of any one of claims 1-12, wherein the method is carried out repeatedly across the cell surface.

14. The method of claim 13, the method further comprising generating a density map of the cell surface molecule on the cell surface.

15. The method of any one of claim 1-14, wherein the cell surface molecule is identified as a cell surface target if the adhesion force measured in (iii) is above a predetermined value.

16. The method of claim 15, wherein the predetermined value is 100 pN.

17. The method of any one of claim 1-14, wherein the cell surface molecule is identified as a cell surface target if the adhesion force measured in (iii) is 100-500 pN more than a control adhesion force.

18. The method of claim 17, wherein the cell surface molecule is identified as a target for in vivo cancer-specific drug delivery if the adhesion force measured in (iii) is at least 400 pN more than a control adhesion force.

19. The method of claim 18, wherein the cell surface molecule is identified as a target for in vivo cancer-specific drug delivery if the adhesion force measured in (iii) is 427 pN more than a control adhesion force.

20. The method of any one of claims 17-19, wherein the control adhesion force is the adhesion force measured using an AFM probe functionalized with a non-specific ligand.

21. The method of claim 20, wherein the non-specific ligand is a non-specific IgG.

22. The method of any one of 1-21, wherein the cell surface molecule is not overexpressed intracellularly or on cell surface.

23. The method of any one of claims 1-22, wherein the AFM probe is functionalized with a plurality of ligands that each associates with a different cell surface molecule of the cell.
